(19) United States
(12) Patent Application Publication
(10) Pub. No.: US 2025/0215370 A1
(43) Pub. Date: Jul. 3, 2025

(54) CELL CULTURE BAG

(71) Applicant: NAN YA PLASTICS CORPORATION, TAIPEI (TW)

(72) Inventors: TE-CHAO LIAO, TAIPEI (TW); Min-Fan Chung, Taoyuan City (TW); CHING-YAO YUAN, TAIPEI (TW)

(21) Appl. No.: 18/610,206

(22) Filed: Mar. 19, 2024

(30) Foreign Application Priority Data
Dec. 27, 2023 (TW) 112150899

(51) Int. Cl. C12M 1/00

(52) U.S. Cl. CPC C12M 23/14; C12M 29/04

(57) ABSTRACT

A cell culture bag is provided. The cell culture bag includes a plastic bag body and at least two connectors disposed on the plastic bag body. A culture space and an external environment are in fluid communication by the connectors. The cell culture bag is formed from a double layer membrane, and a material of the double layer membrane is a polyolefin. The double layer membrane includes a first layer and a second layer, and a melting point of the first layer is higher than a melting point of the second layer. The plastic bag body includes a culture space and a gas permeable surface. The culture space is formed in the plastic bag body. The second layer faces toward the culture space. The culture space and an external environment are in gas communication via the gas permeable surface.

### CROSS-REFERENCE TO RELATED PATENT APPLICATION

This application claims the benefit of priority to Taiwan Patent Application No. 112150899, filed on Dec. 27, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a cell culture bag, and more particularly to a cell culture bag that has excellent gas permeability.

### BACKGROUND OF THE DISCLOSURE

During cell therapy, cells harvested from patients are cultured in a cell culture bag. After adequate proliferation, cultured cells are taken out and then injected into the human body.

Oxygen is needed during a cell growth period, and carbon dioxide needs to be discharged. Therefore, in order to provide a suitable growth environment, a culture flask cannot be full of liquid so as to accommodate sufficient gas. In addition, a liquid level in the culture flask should not be higher than a bottle mouth so as to prevent the liquid from flowing out.

For exchanging gas, a gas permeable film is attached to the bottle mouth of the culture flask, and the culture flask is placed in an incubator. Gas can pass through the gas permeable film. Accordingly, the atmosphere in the culture flask can be controlled by adjusting gas environment conditions in the incubator, so as to control an environment for cell growth.

Since a volume of the culture flask is relatively large, the limitations of the above architecture can cause inconveniences in use. Therefore, how to improve said architecture to overcome the disadvantage resulting from the large volume of the culture flask has become one of the important issues to be addressed in the relevant industry.

### SUMMARY OF THE DISCLOSURE

In response to the above-referenced technical inadequacy, the present disclosure provides a cell culture bag.

In order to solve the above-mentioned problems, one of the technical aspects adopted by the present disclosure is to provide a cell culture bag. The cell culture bag includes a plastic bag body and at least two connectors disposed on the plastic bag body. A culture space and the external environment are optionally in fluid communication via the connectors. The cell culture bag is formed from a double layer membrane, and a material of the cell culture bag is a polyolefin. The double layer membrane includes a first layer and a second layer, a melting point of the first layer is higher than a melting point of the second layer. The plastic bag body includes the culture space formed in the plastic bag body and a gas permeable surface. The culture space is formed in the plastic bag body. The second layer faces toward the culture space. The culture space and an external environment are in gas communication via the gas permeable surface. An average area of the gas permeable surface per unit volume of the culture space ranges from 0.6 cm²/ml to 1.5 cm²/ml. An oxygen transmission rate of the cell culture bag is higher than 200 g/(m²·day), and a water vapor transmission rate of the cell culture bag is lower than 10 g/(m²·day) measured at an environment of 37° C. and 1 atm.

In one of the possible or preferred embodiments, a thickness of the double layer membrane ranges from 50 μm to 140 μm.

In one of the possible or preferred embodiments, a ratio of a thickness of the first layer to a thickness of the second layer ranges from 6 to 12.

In one of the possible or preferred embodiments, the cell culture bag further includes an edge sealing section formed by heat sealing the second layer. A boundary of the culture space is defined by the edge sealing section, and the gas permeable surface is surrounded by the edge sealing section.

In one of the possible or preferred embodiments, a width of the edge sealing section ranges from 6 mm to 15 mm.

In one of the possible or preferred embodiments, a material of the first layer is a linear low density polyethylene which has a melting point ranging from 116° C. to 125° C.

In one of the possible or preferred embodiments, a weight average molecular weight of the linear low density polyethylene ranges from 132,000 g/mol to 134,000 g/mol.

In one of the possible or preferred embodiments, a material of the second layer is a low density polyethylene which has a melting point ranging from 106° C. to 115° C.

In one of the possible or preferred embodiments, a weight average molecular weight of the low density polyethylene ranges from 129,000 g/mol to 131,000 g/mol.

In one of the possible or preferred embodiments, the culture space is used to accommodate a culture medium, and an average area of the gas permeable surface per unit volume of the culture medium ranges from 1.0 cm²/ml to 1.9 cm²/ml.

In one of the possible or preferred embodiments, a total volume of the culture space ranges from 1,000 ml to 2,000 ml, and a total volume of the culture medium ranges from 700 ml to 1,200 ml.

In another aspect, the present disclosure provides a cell culture bag. The cell culture bag includes a plastic bag body and at least two connectors disposed on the plastic bag body. A culture space and the external environment are optionally in fluid communication via the connectors. A material of the cell culture bag is selected from the group consisting of: an ethylene-vinyl acetate copolymer (EVA), a linear low density polyethylene (LLDPE), and a low density polyethylene (LDPE). The plastic bag body includes the culture space formed in the plastic bag body and a gas permeable surface. The culture space is formed in the plastic bag body. The culture space and an external environment are in gas communication via the gas permeable surface. An average area of the gas permeable surface per unit volume of the culture space ranges from 0.6 cm²/ml to 1.5 cm²/ml. An oxygen transmission rate of the cell culture bag is higher than 200 g/(m²·day), and a water vapor transmission rate of the cell culture bag is lower than 10 g/(m²·day) measured at an environment of 37° C. and 1 atm.

In one of the possible or preferred embodiments, a material of the plastic bag body is the ethylene vinyl acetate copolymer which has a melting point ranging from 80° C. to 90° C.

In one of the possible or preferred embodiments, a weight average molecular weight of the ethylene vinyl acetate copolymer ranges from 31,000 g/mol to 33,000 g/mol.

Therefore, in the cell culture bag provided by the present disclosure, by virtue of “the material of the plastic bag body” and “the average area of the gas permeable surface per unit volume of the culture space ranging from 0.6 cm²/ml to 1.5 cm²/ml,” the cell culture bag can have good oxygen transmission rate and an appropriate water vapor transmission rate.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic side view of a cell culture bag according to the present disclosure;

FIG. 2 is a schematic cross-sectional view of the cell culture bag according to one embodiment of the present disclosure taken along line II-II of FIG. 1; and

FIG. 3 is a schematic cross-sectional view of the cell culture bag according to another embodiment of the present disclosure taken along line II-II of FIG. 1.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

In order to improve the problem of a large size of the conventional culture flask, the present disclosure provides a cell culture bag. The cell culture bag has good gas permeability, in which gas can directly be exchanged with gas from the external environment. Therefore, the problem of a large size for the accommodation of gas existing in the conventional culture flask can be solved, and the cell culture bag of the present disclosure has an advantage of convenience in use.

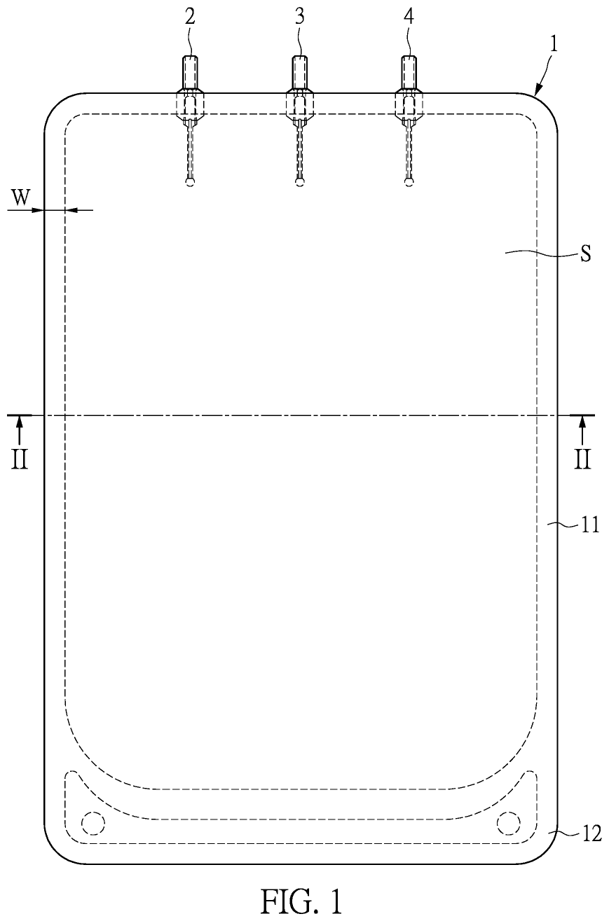

Referring to FIG. 1, the cell culture bag of the present disclosure has a plastic bag body 1 and at least two connectors disposed on the plastic bag body 1. Types of the connectors can be chosen according to various purposes, such as injection, sampling, or discharge of a culture medium. For convenience of illustration, the connectors shown in FIG. 1 can include an injection connector 2, a sample connector 3, and a discharge connector 4.

For example, the injection connector 2 and the discharge connector 4 can be a needle-free connector, and the sample connector 3 can be a male luer, but the present disclosure is not limited thereto. In practical operation, once the connector is used, the connector needs to be cut to avoid pollution.

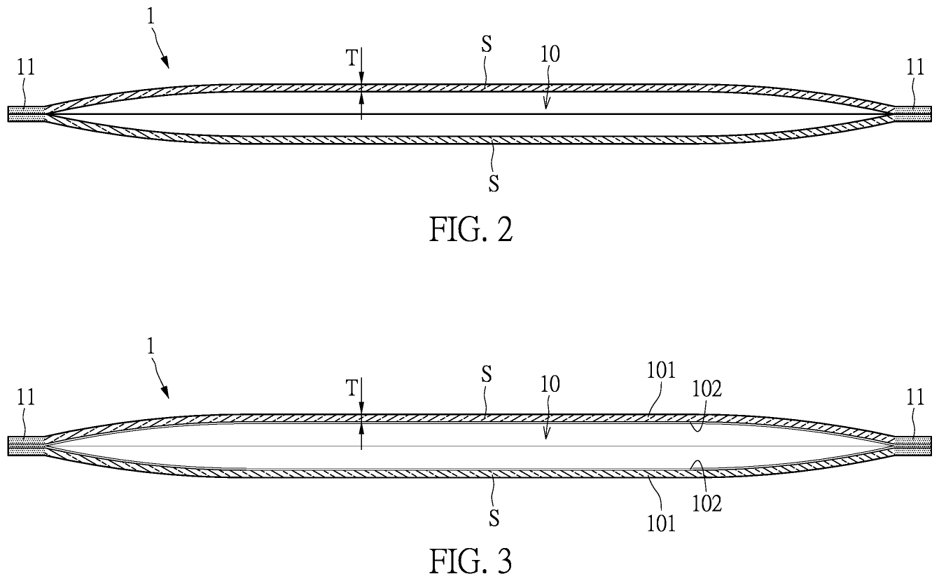

Referring to FIG. 1 and FIG. 2, the plastic bag body 1 is a light-transmitting bag body such as to observe the status of the cells in a culture space 10. Specifically, a transmittance of the plastic bag body 1 is higher than 90%, and a haze of the plastic bag body 1 is lower than 11%.

The culture space 10 is formed in the plastic bag body 1. The culture space 10 can be used to accommodate a culture medium, so as to provide cells with a sterile environment for growth. The culture space 10 and the external environment are fluidly communicated with each other via the connectors. Hence, it is convenient for the injection, sampling, or discharge of the culture medium.

In order to form the culture space 10, a membrane material is folded in half, and undergoes a heat sealing process. The part of the membrane material undergoing the heat sealing process is melted and then connected with the other half of the folded membrane material, such that an edge sealing section 11 is formed on the plastic bag body 1.

Except for the locations of the connectors, a closed area is formed by the edge sealing section 11, and a range of the culture space 10 is defined by the edge sealing section 11. The larger the closed area formed by the edge sealing section 11 is, the larger the culture space 10 is. Similarly, the smaller the closed area formed by the edge sealing section 1 is, the smaller the culture space 10 is. In an exemplary embodiment, a volume of the culture space 10 ranges from 1000 ml to 2000 ml, but the present disclosure is not limited thereto. In an exemplary embodiment, a width of the edge sealing section 11 ranges from 6 mm to 15 mm, but the present disclosure is not limited thereto.

Holes can be formed on the plastic bag body 1, such that the plastic bag body 1 can be used in a hanging state. In order to prevent the culture medium from leaking from the holes in the hanging state, a leakage-proof section 12 can be further formed on the plastic bag body 1. In an exemplary embodiment, the leakage-proof section 12 and the edge sealing section 11 are integrally formed, and the holes are surrounded by the edge sealing section 11 and the leakage-proof section 12. Therefore, the holes are not fluidly communicated with the culture space 10.

In other exemplary embodiments, the leakage-proof section 12 can be independently formed on the plastic bag body 1, and the holes can be surrounded by the leakage-proof section 12, but the present disclosure is not limited thereto.

During the cell growth period, cells need oxygen and discharge carbon dioxide. Hence, the plastic bag body 1 is formed from a gas permeable material. Specifically, both sides of the plastic bag body 1 have a gas permeable surface S, and the gas permeable surface S is surrounded by the edge sealing section 11. The two gas permeable surfaces S are located on two opposite sides of the culture space 10. In this way, the culture space 10 and the external environment can be in a gas communication via the gas permeable surface S for gas exchange.

In order to avoid cell death for lack of oxygen caused by insufficient ventilation rate, an average area of the gas permeable surface S per unit volume of the culture space 10 is controlled to range from 0.6 cm²/ml to 1.5 cm²/ml in the present disclosure. For example, the average area of the gas permeable surface S per unit volume of the culture space 10 can be 0.8 cm²/ml, 1.0 cm²/ml, 1.2 cm²/ml, or 1.4 cm²/ml. Preferably, the average area of the gas permeable surface S per unit volume of the culture space 10 can range from 0.65 cm²/ml to 1.35 cm²/ml.

In an exemplary embodiment, a total area of both the gas permeable surfaces S is 1314 cm². When the culture space 10 is 1000 ml, the average area of the gas permeable surface S per unit volume of the culture space 10 is 1.314 cm²/ml. When the culture space 10 is 2000 ml, the average area of the gas permeable surface S per unit volume of the culture space 10 is 0.657 cm²/ml.

In order to enhance convenience of use and a cell survival rate, the culture space 10 will not be full of the culture medium. For example, the culture space 10 can be filled with the culture medium to 50% to 75% full (i.e., leaving 25% to 50% empty space). In an exemplary embodiment, an added amount of the culture medium can range from 700 ml to 1200 ml, but the present disclosure is not limited thereto.

According to experimental results, when an average area of the gas permeable surface S per unit volume of the culture medium ranges from 1.0 cm²/ml to 1.9 cm²/ml, the cell survival rate can be maintained without enlarging the volume of the cell culture bag 1. For example, the average area of the gas permeable surface S per unit volume of the culture medium can be 1.0 cm²/ml, 1.2 cm²/ml, 1.4 cm²/ml, 1.6 cm²/ml, or 1.8 cm²/ml.

In an exemplary embodiment, a total area of both the gas permeable surfaces S is 1314 cm². When the added amount of the culture medium is 700 ml, the average area of the gas permeable surface S per unit volume of the culture medium is 1.877 cm²/ml. When the added amount of the culture medium is 1200 ml, the average area of the gas permeable surface S per unit volume of the culture medium is 1.095 cm²/ml.

In order to enhance the gas permeability of the cell culture bag 1, a material of the cell culture bag 1 can include at least one of an ethylene-vinyl acetate copolymer (EVA), a linear low density polyethylene (LLDPE), and a low density polyethylene (LDPE).

Specifically, a melting point of the ethylene-vinyl acetate copolymer measured by differential scanning calorimetry (DSC) ranges from 80° C. to 90° C. Preferably, the melting point of the ethylene-vinyl acetate copolymer ranges from 82° C. to 86° C. A weight average molecular weight of the ethylene-vinyl acetate copolymer measured by gel permeation chromatography (GPC) ranges from 31,000 g/mol to 33,000 g/mol. Preferably, the weight average molecular weight of the ethylene-vinyl acetate copolymer ranges from 31,500 g/mol to 32,500 g/mol. A melting index of the ethylene-vinyl acetate copolymer measured according to the ASTM D1238 standard ranges from 1.6 to 2.0.

Preferably, the melting index of the ethylene-vinyl acetate copolymer ranges from 1.7 to 1.9. A density of the ethylene-vinyl acetate copolymer measured according to the ASTM D1505 standard ranges from 0.910 g/cm³ to 0.950 g/cm³. Preferably, the density of the ethylene-vinyl acetate copolymer ranges from 0.935 g/cm³ to 0.940 g/cm³.

Specifically, a melting point of the linear low density polyethylene measured by DSC ranges from 116° C. to 125° C. Preferably, the melting point of the linear low density polyethylene ranges from 120° C. to 124° C. A weight average molecular weight of the linear low density polyethylene measured by GPC ranges from 132,000 g/mol to 134,000 g/mol. Preferably, the weight average molecular weight of the linear low density polyethylene ranges from 132,500 g/mol to 133,500 g/mol. A melting index of the linear low density polyethylene measured according to the ASTM D1238 standard ranges from 1.8 to 2.2. Preferably, the melting index of the linear low density polyethylene ranges from 1.9 to 2.1. A density of the linear low density polyethylene measured according to the ASTM D1505 standard ranges from 0.910 g/cm³ to 0.950 g/cm³. Preferably, the density of the linear low density polyethylene ranges from 0.915 g/cm³ to 0.925 g/cm³.

Specifically, a melting point of the low density polyethylene measured by DSC ranges from 106° C. to 115° C. Preferably, the melting point of the low density polyethylene ranges from 109° C. to 113° C. A weight average molecular weight of the low density polyethylene measured by GPC ranges from 129,000 g/mol to 131,000 g/mol. Preferably, the weight average molecular weight of the low density polyethylene ranges from 129,500 g/mol to 130,500 g/mol. A melting index of the low density polyethylene measured according to the ASTM D1238 standard ranges from 1.8 to 2.2. Preferably, the melting index of the low density polyethylene ranges from 1.9 to 2.1. A density of the low density polyethylene measured according to the ASTM D1505 standard ranges from 0.910 g/cm³ to 0.950 g/cm³. Preferably, the density of the low density polyethylene ranges from 0.920 g/cm³ to 0.925 g/cm³.

Referring to FIG. 2, a thickness T of the cell culture bag 1 also influences the gas permeability. When the thickness T of the cell culture bag 1 ranges from 50 μm to 140 μm, the cell culture bag 1 can have good gas permeability. Specific experimental results are described later.

By choosing the specific material and adjusting the thickness, the cell culture bag can have an oxygen transmission rate higher than 200 g/(m²·day) measured at an environment of 37° C. and 1 atm and a water vapor transmission rate lower than 10 g/(m²·day) measured at an environment of 37° C. and 1 atm.

In order to prove that the cell culture bag of the present disclosure can have good gas permeability, the ethylene-vinyl acetate copolymer, the linear low density polyethylene, and the low density polyethylene are respectively used to manufacture the cell culture bags having different thicknesses (Test 1 to Test 9). Properties of the cell culture bags are measured, and the results are listed in Table 1.

In Table 1, the water vapor transmission rate of the cell culture bag is measured according to the ASTM F1249 standard at an environment of 37° C. and 1 atm. The oxygen transmission rate of the cell culture bag is measured according to the ASTM D3985 standard at an environment of 37° C. and 1 atm. The transmittance and the haze of the cell culture bag are measured by a haze meter according to the ASTM D1003 standard. The surface resistance of the cell culture bag is measured by a surface resistance meter. The tensile strength of the cell culture bag is measured by a universal testing machine according to the ASTM D882 standard.

TABLE 1

| | | | Water vapor | Oxygen | | | Surface | Tensile |
|---|---|---|---|---|---|---|---|---|
| | | Thickness | transmission rate | transmission rate | Transmittance | Haze | impedance | strength |
| | Material | (μm) | (g/(m² · day)) | (g/(m² · day)) | (%) | (%) | (—) | (kgf) |
| Test 1 | LLDPE | 150 | 2.21 | 127 | 91.11 | 7.99 | >2 × 10¹² | 3.91 |
| Test 2 | | 100 | 3.95 | >200 | 92.05 | 6.78 | | 3.51 |
| Test 3 | | 80 | 4.66 | >200 | 92.57 | 6.11 | | 3.12 |
| Test 4 | LDPE | 150 | 2.54 | 157 | 90.95 | 8.34 | | 4.91 |
| Test 5 | | 100 | 4.03 | >200 | 91.95 | 7.67 | | 4.21 |
| Test 6 | | 80 | 5.72 | >200 | 92.13 | 7.17 | | 3.92 |
| Test 7 | EVA | 130 | 4.59 | >200 | 91.25 | 10.81 | | 4.48 |
| Test 8 | | 100 | 6.59 | >200 | 92.01 | 9.14 | | 3.99 |
| Test 9 | | 80 | 7.59 | >200 | 92.55 | 8.10 | | 3.08 |

According to the results in Table 1, when the cell culture bag is too thick, the oxygen transmission rate is decreased and cannot reach the expected value (higher than 200 g/m²/day). When the cell culture bag is too thin, even though the oxygen transmission rate is sufficient for cell growth, it is accompanied by an increased water vapor transmission rate. If the water evaporates too fast, a concentration of the culture medium will increase, which is likely to lead to death of cells. Therefore, under a comprehensive evaluation of the oxygen transmission rate and the water vapor transmission rate, the thickness of the cell culture bag preferably ranges from 50 μm to 140 μm.

According to the results in Table 1, when the material of the cell culture bag is selected from the group consisting of the ethylene-vinyl acetate copolymer, the linear low density polyethylene, and the low density polyethylene. The cell culture bag can have appropriate oxygen transmission rate and water vapor transmission rate. Moreover, the cell culture bag can have appropriate transmittance (higher than 90%) and haze (lower than 11%).

In addition, the tensile strength of the cell culture bag can be higher than 3 kgf. Preferably, the tensile strength of the cell culture bag can range from 3 kgf to 5.5 kgf. Therefore, the cell culture bag will not be easily cleaved by external forces during the processing operation. The surface resistance of the culture medium can be used to evaluate the cell growth status. Therefore, the surface resistance of the cell culture bag needs to be higher than 2×10¹² so as not to disturb the measurement of the surface resistance of the culture medium.

The present disclosure provides another cell culture bag of another embodiment whose structure is similar to the cell culture bag shown in FIG. 1. The cell culture bag has the plastic bag body 1, and the injection connector 2, the sampling connector 3, and the discharge connector 4 disposed on the plastic bag body 1. The difference is that the plastic bag body 1 is formed from a double layer membrane.

Referring to FIG. 3, the double layer membrane undergoes a heat sealing process. The part of the double layer membrane undergoing the heat sealing process is melted and then connected with the other half of the folded membrane material, such that the edge sealing section 11 is formed on the plastic bag body 1. A range of the culture space 10 is defined by the edge sealing section 11, and the gas permeable surface S is surrounded by the edge sealing section 11. In an exemplary embodiment, a thickness of the double layer membrane ranges from 50 μm to 140 μm.

Specifically, the double layer membrane includes a first layer 101 and a second layer 102. The first layer 101 is disposed on the second layer 102. The second layer 102 is located inside of the plastic bag body 1. In other words, the second layer 102 faces toward the culture space 10. Therefore, during the heat sealing process, a part of the second layer 102 is melting and connected to form the edge sealing section 11.

In order to achieve the expected gas permeability, the thickness T of the double layer membrane cannot be higher than 150 μm. However, the double layer membrane may face the problem of cleavage during the heat sealing process. For solving this problem, composite materials that have different melting points are used as the double layer membrane. A melting point of the first layer 101 is higher than a melting point of the second layer 102. Accordingly, the double layer membrane can be heat sealed by a lower temperature to form the edge sealing section 11, thereby avoiding the membrane cleavage.

The difference in the melting points allows the double layer membrane to have the good gas permeability and a low processing temperature. In an exemplary embodiment, a material of the first layer 101 is a linear low density polyethylene, and a material of the second layer 102 is a low density polyethylene. The materials of the first layer 101 and the second layer 102 belong to similar types, such that they can be co-extruded to form the double layer membrane. The difference between the first layer 101 and the second layer 102 is the melting point. The melting point of the first layer 101 is higher than the melting point of second layer 102 by 3° C. to 20° C. Preferably, the melting point of the first layer 101 is higher than the melting point of the second layer 102 by 5° C. to 15° C.

According to experimental results, when a ratio of a thickness of the first layer to a thickness of the second layer ranges from 6 to 12, the double layer membrane can have good gas permeability and heat sealing ability. Preferably, the ratio of a thickness of the first layer to a thickness of the second layer ranges from 8 to 10.

In order to prove that the cell culture bag of the present disclosure has good gas permeability, the linear low density polyethylene and the low density polyethylene are co-extruded to form the double layer membrane having a thickness of 100 μm. The double layer membrane is used to manufacture the plastic bag body (Test 10 to Test 12). In Test 10 to Test 12, the double layer membranes have different thickness ratios of the first layer 101 to the second layer 102.

The plastic bag body undergoes a water vapor transmission rate test, an oxygen transmission rate, a transmittance test, a haze test, a surface resistance test, a tensile strength test, and a bag breakage rate test, and the results are listed in Table 2. The water vapor transmission rate test, the oxygen transmission rate, the transmittance test, the haze test, the surface resistance test, and the tensile strength test are the same with those mentioned above, and are not reiterated herein.

In the bag breakage rate test, the plastic bag body is heat sealed at 115° C. to manufacture the cell culture bag. Subsequently, the culture medium is filled in the cell culture bag to 70% full, and then a 1 kg weight object is placed on the cell culture bag to observe whether the cell culture bag is broken or not. The steps mentioned above, i.e., filling the culture medium and placing a 1 kg weight object, are conducted multiple times so as to count the bag breakage rate. For example, a bag breakage rate of 15% means that three cell culture bags are broken out of twenty cell culture bags.

TABLE 2

| | Thickness | | | | | | | |
|---|---|---|---|---|---|---|---|---|
| | ratio of | Water vapor | Oxygen | | | | | |
| | first layer | transmission | transmission | | | Surface | Tensile | Bag |
| | to second | rate | rate | Transmittance | Haze | impedance | strength | breakage |
| | layer | (g/(m² · day)) | (g/(m² · day)) | (%) | (%) | (—) | (kgf) | rate |
| Test | 0:10 | 4.03 | >200 | 91.95 | 7.67 | >2 × 10¹² | 4.21 | 50% |
| 10 | | | | | | | | |
| Test | 9:1 | 3.27 | >200 | 92.11 | 6.99 | | 3.87 | 15% |
| 11 | | | | | | | | |
| Test | 8:2 | 4.17 | >200 | 91.00 | 7.99 | | 3.99 | 30% |
| 12 | | | | | | | | |
| Test | 5:5 | 3.99 | >200 | 91.35 | 7.69 | | 4.31 | 40% |
| 13 | | | | | | | | |

According to the results in Table 2, when the ratio of the first layer to the second layer is 9:1, the cell culture bag has the lowest bag breakage rate. In addition, the bag breakage rate of the cell culture bag will increase as the thickness of the second layer.

According to the results in Table 1 and Table 2, the first layer and the second layer enable the cell culture bag to have the expected oxygen transmission rate and water vapor transmission rate, and further have a good heat sealing ability, thus being unlikely to break.

### Beneficial Effects of the Embodiments

In conclusion, in the cell culture bag provided by the present disclosure, by virtue of “the material of the plastic bag body” and “an average area of the gas permeable surface per unit volume of the culture space ranging from 0.6 cm²/ml to 1.5 cm²/ml,” the cell culture bag can have good oxygen transmission rate and an appropriate water vapor transmission rate.

Further, in order to improve a manufacturing yield of the cell culture bag on a condition of maintaining appropriate gas transmission rates, the double layer membrane can be used to manufacture the cell culture bag. By laminating two layers that have different melting points, the processing temperature during the heat sealing process can be decreased, such that the double layer membrane is unlikely to be broken during the heat sealing process.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A cell culture bag, comprising:
a plastic bag body having a polyolefin material and being formed from a double layer membrane, wherein the double layer membrane includes a first layer and a second layer, a melting point of the first layer is higher than a melting point of the second layer, and the plastic bag body includes:
a culture space formed in the plastic bag body, wherein the second layer faces toward the culture space; and
a gas permeable surface, wherein the culture space and an external environment are in gas communication via the gas permeable surface;
wherein an average area of the gas permeable surface per unit volume of the culture space ranges from 0.6 cm²/ml to 1.5 cm²/ml; and
at least two connectors disposed on the plastic bag body so as to selectively put the culture space and the external environment in fluid communication;
wherein an oxygen transmission rate of the cell culture bag is higher than 200 g/(m²·day), and a water vapor transmission rate of the cell culture bag is lower than 10 g/(m²·day).

2. The cell culture bag according to claim 1, wherein a thickness of the double layer membrane ranges from 50 μm to 140 μm.

3. The cell culture bag according to claim 1, wherein a ratio of a thickness of the first layer to a thickness of the second layer ranges from 6 to 12.

4. The cell culture bag according to claim 1, further comprising an edge sealing section formed by heat sealing the second layer; wherein a boundary of the culture space is defined by the edge sealing section, and the gas permeable surface is surrounded by the edge sealing section.

5. The cell culture bag according to claim 4, wherein a width of the edge sealing section ranges from 6 mm to 15 mm.

6. The cell culture bag according to claim 1, wherein a material of the first layer is a linear low density polyethylene which has a melting point ranging from 116° C. to 125° C.

7. The cell culture bag according to claim 6, wherein a weight average molecular weight of the linear low density polyethylene ranges from 132,000 g/mol to 134,000 g/mol.

8. The cell culture bag according to claim 1, wherein a material of the second layer is a low density polyethylene which has a melting point ranging from 106° C. to 115° C.

9. The cell culture bag according to claim 8, wherein a weight average molecular weight of the low density polyethylene ranges from 129,000 g/mol to 131,000 g/mol.

10. The cell culture bag according to claim 1, wherein the culture space is used to accommodate a culture medium, and an average area of the gas permeable surface per unit volume of the culture medium ranges from 1.0 cm²/ml to 1.9 cm²/ml.

11. The cell culture bag according to claim 10, wherein a total volume of the culture space ranges from 1,000 ml to 2,000 ml, and a total volume of the culture medium ranges from 700 ml to 1,200 ml.

12. A cell culture bag, comprising:
a plastic bag body whose material is selected from the group consisting of: an ethylene vinyl acetate copolymer, a linear low density polyethylene and a low density polyethylene; wherein the plastic bag body includes:
a culture space formed in the plastic bag body; and
a gas permeable surface; wherein the culture space and an external environment are in gas communication via the gas permeable surface;
wherein an average area of the gas permeable surface per unit volume of the culture space ranges from 0.6 cm²/ml to 1.5 cm²/ml; and
at least two connectors disposed on the plastic bag body so as to selectively put the culture space and the external environment in fluid communication;
wherein an oxygen transmission rate of the cell culture bag is higher than 200 g/(m²·day), and a water vapor transmission rate of the cell culture bag is lower than 10 g/(m²·day).

13. The cell culture bag according to claim 12, wherein a material of the plastic bag body is the ethylene vinyl acetate copolymer which has a melting point ranging from 80° C. to 90° C.

14. The cell culture bag according to claim 13, wherein a weight average molecular weight of the ethylene vinyl acetate copolymer ranges from 31,000 g/mol to 33,000 g/mol.
